(19) United States
(12) Patent Application Publication
(10) Pub. No.: US 2005/0106583 A1
(43) Pub. Date: May 19, 2005

(54) Porous device

(76) Inventors: David Cossaboon, Germantown, MD (US); Eric Eastman, Highland, MD (US); Brady Cheek, Walkersville, MD (US); Yong-Yi Yu, Germantown, MD (US)

Correspondence Address: BLANK ROME LLP, 600 NEW HAMPSHIRE AVENUE, N.W., WASHINGTON, DC, 20037, US

(21) Appl. No.: 10/839,175

(22) Filed: May 6, 2004

Related U.S. Application Data
(60) Provisional application No. 60/468,097, filed on May 6, 2003.

(51) Int. Cl. C12Q 1/68; C12M 1/34

(52) U.S. Cl. 435/6; 435/287.2

(57) ABSTRACT

An improved polymer device for conducting a multiplicity of individual and simultaneous binding reactions or performing other separation techniques is described. The apparatus comprises a polymer substrate on which are located discrete and isolated sites for such reactions or separation phenomena. The apparatus is characterized by discrete and isolated regions that extend through said substrate and terminate on a second surface thereof such that when a test sample is allowed to the substrate, it is capable of penetrating through each such region during the course of a reaction. The use of castable polymer techniques improves the manufacturing flexibility and cost over prior materials and fabrication methods and also provides certain improved properties such as lower specific stiffness and ease of lamination or bonding to other elements of a device.

### FIELD OF THE INVENTION

The invention relates to an improved porous device manufactured from a polymer. The device may be used for the detection of binding reactions. The invention also relates to the method of making such a device as well as the various applications and/or uses for such a device. For example, the device may be used not only for the detection of binding reactions, it has various other uses such as for cell separation processes or chromatography.

### BACKGROUND OF THE INVENTION

The benefits of a “Microfabricated, Flowthrough Porous Apparatus for Discrete Detection of Binding Reactions” when compared to flat surface devices are disclosed in U.S. Pat. No. 5,843,767 to Beattie, the entire disclosure of which is incorporated herein by reference. Although this list is comprehensive, it is not necessarily an exhaustive list. Some of the benefits of Beattie's device are (1) improved detection sensitivity due to the vastly increased surface area which increases the quantity of nucleic acid bound per cross sectional area; (2) minimization of a rate-limiting diffusion step preceding the hybridization reaction (reducing the time required for the average target molecule to encounter a surface-tethered probe from hours to milliseconds), speeding hybridization and enabling mismatch discrimination at both forward and reverse reactions; (3) enablement of the analysis of dilute nucleic acid solutions because of the ability to gradually flow the solution through the porous wafer; (4) facilitation of subsequent rounds of hybridization involving delivery of probes to specific sites within the hybridization array; (5) facilitation of the recovery of bound nucleic acids from specific hybridization sites within the array, enabling the further analysis of such recovered molecules; and (6) facilitation of the chemical bonding of probe molecules to the surface within each isolated region due to the avoidance of the rapid drying of small droplets of probe solution on flat surfaces exposed to the atmosphere.

In U.S. Pat. No. 6,225,131 to van Damme, et al the perceived disadvantages of the use of glass or silicon as porous substrate materials for an array device are disclosed. Cost of production and certain physical limitations of those materials are mentioned. The use of an electrochemically manufactured metal oxide material as a porous substrate is proposed as a solution.

Polymers, which are often generically referred to as “plastics”, have historically been used in a multitude of applications where inexpensive materials and/or manufacturing flexibility were a solution to a limitation of the existing material options for a particular device.

In connection with the manufacture of a porous array device from glass or silicon, the cost of manufacture may indeed be much higher than that which could be obtained by the use of polymers. Moreover, certain manufacturing technologies employing silicon have cost factors associated with changing any chip dimensions or configurations.

The use of castable polymers for microfluidic devices has been proposed by Jackman et al., J. Micromech. Microeng. 11 (2001) pp. 1-8.

However, castable polymers have heretofore not been proposed as a substrate material for a porous device having channels capable of supporting liquid flow as are disclosed in the Beattie '767 patent or the van Damme et al '131 patent.

It would be desirable to have the benefits of castable polymer technology in a porous device such as the device disclosed in U.S. Pat. No. 5,843,767 to Beattie. In particular, castable polymers are flexible, and may be bent without fracture, as opposed to more brittle ceramics and glass. Castable polymers may also have low cost, be amenable to different dimensional manufacturing configurations and are more processable than non-polymer materials. For example, polymer materials may be easily laminated to increase thickness.

### SUMMARY OF THE INVENTION

The invention provides the benefits of castable polymer technology in a porous array device having channels such as the device disclosed in U.S. Pat. No. 5,843,767 to Beattie. In particular, castable polymers are used to provide the porous, flexible device in accordance with the invention. The devices in accordance with the invention may be bent without fracture, as opposed to more brittle ceramic and glass materials. This property is called specific stiffness, and is calculated by dividing the modulus of elasticity of the material by its density. The specific stiffness values for the castable polymers in accordance with the invention are lower than that for glass or silicon. The castable polymers in accordance with the invention provide low cost, are amenable to different dimensional manufacturing configurations and are more processable than non-polymer materials. The castable polymers in accordance with the invention may be laminated to increase thickness.

The invention provides a device for binding a target molecule, comprising a polymer substrate having oppositely facing first and second major surfaces; a multiplicity of discrete channels extending through said polymer substrate from said first major surface to said second major surface; a first binding reagent immobilized on the walls of a first group of said channels, and a second binding reagent immobilized on the walls of a second group of said channels. In embodiments of the invention, said first and second binding reagents differ from one another. In other embodiments of the invention, the first and second binding reagents bind different target molecules.

### BRIEF DESCRIPTION OF THE DRAWINGS

FIG. 1 shows a plan view of a proposed configuration of a device in accordance with the invention, including an expanded view of the porous portion.

FIG. 2 is a reproduction of scanning electron microscope images of a device manufactured from SU-8 polymer in accordance with the invention.

FIG. 3 is a reproduction of a scanning electron microscope image of a device manufactured from SU-8 polymer in accordance with the invention.

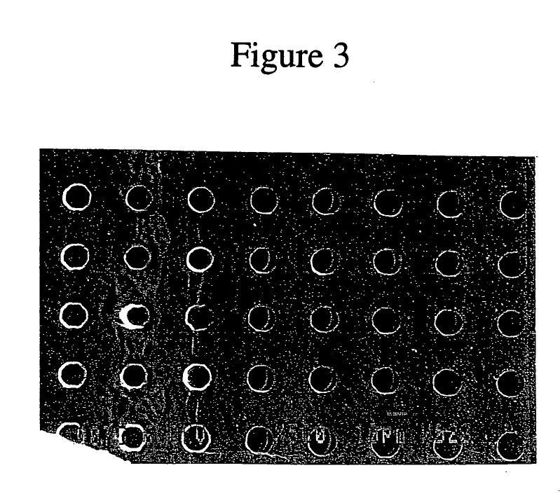

### DETAILED DESCRIPTION OF THE INVENTION

This porous polymer device in accordance with the invention may be employed in a variety of analytical tasks, including nucleic acid sequence analysis by hybridization, analysis of patterns of gene expression by hybridization of cellular MRNA to an array of gene-specific probes, immunochemical analysis of protein mixtures, epitope mapping, assay of receptor-ligand interactions, and profiling of cellular populations involving binding of cell surface molecules to specific ligands or receptors immobilized within individual binding sites. The present invention can be equally applied to a broad range of molecular binding reactions involving small molecules, macromolecules, particles, and cellular systems. Furthermore, the porous polymer device in accordance with the invention may be employed in separation techniques such as chromatography or cell separation processes. In particular, in embodiments of the invention, the separation of red blood cells from plasma may be performed.

In embodiments of the invention, the porous device of the present invention provides a novel flow-through “genosensor”, in which nucleic acid recognition elements are immobilized within densely packed pores or channels, arranged in patches across a wafer of the polymer support material. Flow-through “genosensors” in accordance with the invention may utilize a variety of conventional detection methods, including microfabricated optical and electronic detection components, film, charge-coupled-device arrays, camera systems and phosphor storage technology.

In embodiments of the invention, the porous device of the present invention provides for the conduction and detection of binding reactions. In particular, the present invention provides improved “genosensors” and methods for the use thereof in the identification or characterization of nucleic acid sequences through nucleic acid probe hybridization with samples containing an uncharacterized polynucleic acid, e.g., a cDNA, MRNA, recombinant DNA, polymerase chain reaction (PCR) fragments or the like, as well as other biomolecules.

### Castable Polymers

There are many castable polymers available for use. Acrylics, polyurethanes, fluoropolymers, silicone polymers and epoxies are all castable polymers in the context of the invention. Epoxy based polymers are the preferred castable polymers in accordance with the invention. Epoxy based photo-imagable polymers have been extensively developed in the electronics industry because of their use in circuit board manufacture. In particular, the most preferred castable polymer in accordance with the invention is designated “SU-8”, a product developed and patented by IBM Corporation. SU-8 is described in U.S. Pat. No. 4,882,245, the disclosure of which is incorporated by reference herein in its entirety. Another castable epoxy composition useful in the present invention is described in U.S. Pat. No. 5,304,457, the disclosure of which is incorporated by reference herein in its entirety. SU-8 is a negative, epoxy-based, near-UV photoresist composition and is particularly desirable in that it can be used to form high aspect ratio microstructures which are required for the vertical capillaries in the porous device in accordance with the invention. SU-8 also has excellent chemical resistance and is a biocompatible material, supporting molecular attachment of the binding reagents in accordance with the invention. SU-8 has been licensed by IBM to two companies. MicroChem. Inc. of Newton, Mass. sells various formulations of SU-8 having different viscosities under the designations SU-8 (5); SU-8 (10); SU-8 (25); SU-8 (50) and SU-8 (100). SU-8 (2000) is also available from MicroChem wherein the standard GBL solvent has been replaced by cyclopentanone. Sotec Microsystem of Renens, Switzerland also sells SU-8 compositions of varying viscosities under license under the product designations SM1040, SM1060 and SM1070. Other versions of the product with a low coefficient of thermal expansion are available.

### The Casting Process

The porous device in accordance with the invention is made by a casting process using a castable polymer. In the first step of the casting process, a reverse of the desired porous device (or a “mold”) is etched into a silicon wafer using a reactive ion etching (“RIE”) process. Preferably, a patterned silicon mold is formed in this manner. Other methods known in the art such as lithography or electroforming may be used to form a mold from other materials. A very thin layer of release material may be deposited on the patterned silicon mold to facilitate the separation between the cast porous device of the invention and the patterned silicon mold. The castable polymer is then applied to the patterned silicon mold in the desired thickness. Spin coating, or injection, or other conventional polymer application methods may be used. After the castable polymer has sufficiently conformed to the patterned silicon mold and has the desired thickness, as mentioned above, near UV wavelength radiation is used to cure and/or crosslink the castable polymer. In embodiments of the invention, UV exposure may occur in a pressurized mold, an autoclave, an oven, or the like. To facilitate removal of the cured device from the mold, a photomask may be used during UV exposure to prevent curing immediately adjacent the pillar structure in the mold for what will be each microchannel in the porous device of the invention.

An example of a microstructure molding process is described in U.S. Pat. No. 6,454,970, the disclosure of which is incorporated by reference herein in its entirety.

Attachment Chemistry

The porous device in accordance with the invention may be functionalized for DNA or protein attachment via epoxy silane methods, dextran with epoxide/aldehyde/carboxy terminus, by direct post cure activation of the cured, castable polymer, or by other methods known in the art. In addition, a metallic coating of a noble metal, for example gold, may be applied to the porous device of the invention. Sputtering or other available methods may be used to apply this coating. The gold coating may be used to vary the opacity of the porous device or to facilitate other conventional molecular attachment methods which may be employed in accordance with the invention.

Certain castable polymers are hydrophobic and a hydrophilic coating may be applied to the porous device in accordance with the invention in order to facilitate spotting or printing as well as the performance of the assay itself.

Attachment chemistry using dextran-agarose supports is described in Penzol et al, Biotechnol. Bioeng. 60 (1998) pp. 518-523.

Binding reagents may be applied to a particular region of the porous device in one step or may be synthesized in situ as is well known in the art. Ink-jet technology may be used to accurately deposit materials in a predetermined pattern as is well known in the art and described in the Beattie '767 patent and the van Damme '131 patent.

Hybridization or binding conditions and/or techniques vary according to the specific probes and/or target molecules and are well known in the art.

Dimensions

In embodiments of the invention, the porous device of the invention may have discrete channels having diameters of from about 0.033 micrometers to about 10 micrometers. In other embodiments of the invention, diameters on the order of from 10 to 75 micrometers may be used in order to accommodate other processes that require large microchannel diameters such as blood separation processes. In embodiments of the invention, the porous device of the invention may have discrete channels having cross sectional areas of between about 8.5×10⁻⁴ μm² to 80 μm². In embodiments of the invention, the porous device of the invention may have substrate thickness between about 100 μm to about 1000 μm thick. Lamination of individual porous devices to form thicker substrates may be used if the casting process is limited in the thickness that may be achieved in a single casting. In embodiments of the invention, the porous device of the invention may have channels having an inner surface area of between about 10 m and about 3 μm².

The porous device for binding a target molecule in accordance with the invention has a multiplicity of discrete channels extending through said polymer substrate from the first major surface to said second major surface and a first binding reagent immobilized on the walls of a first group of channels and a second binding reagent immobilized on the walls of a second group of channels.

In embodiments of the invention, the porous device of the invention may have groups of channels having areas of between about 20 μm² to about 3 μm². In embodiments of the invention, the porous device of the invention may have between 400 and 4400 of the groups of discrete channels per cm² of cross-sectional area of the substrate. In embodiments of the invention, the porous device of the invention may have the inner surface area of the channels in a group of channels from about 100 to about 1000 times the cross sectional area of the group of channels.

In embodiments of the invention, the channels may be either round or square in cross section. The pitch between the channels (center to center distance) may be from about 5 micrometers to about 75 micrometers, preferably from about 8 micrometers to about 15 micrometers.

In embodiments of the invention, the aspect ratio, the ratio of the height of the channels to their width or diameter, is greater than about 10, preferably greater than about 15 and more preferably greater than about 20.

In embodiments of the invention, the substrate after curing is opaque to visible light, including inside the channels.

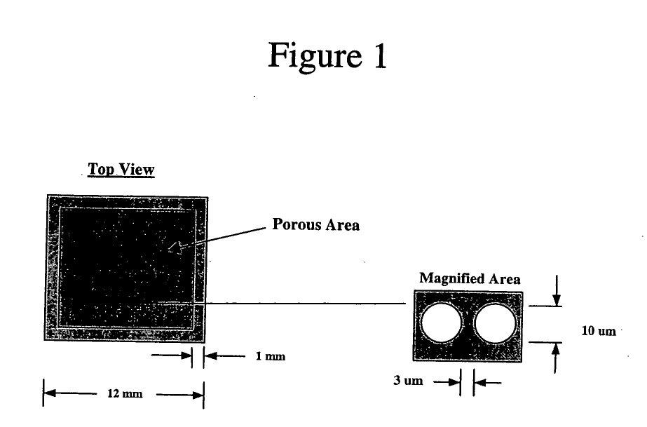

As is shown in FIG. 1, the device may have a porous area and a non-porous border area. There may be on the order of 500,000 to 1,006,000 channels in the porous area defined by the 10 mm by 10 mm dimension in FIG. 1. Approximately 600,000 is a preferred number of channels. In the particular example shown in FIG. 1, the thickness of the porous and non-porous area is 100 μm and is the same, although it may be different in other embodiments of the invention. The diameter of the channels in FIG. 1 is 10 μm and the space between channels is 3 μm.

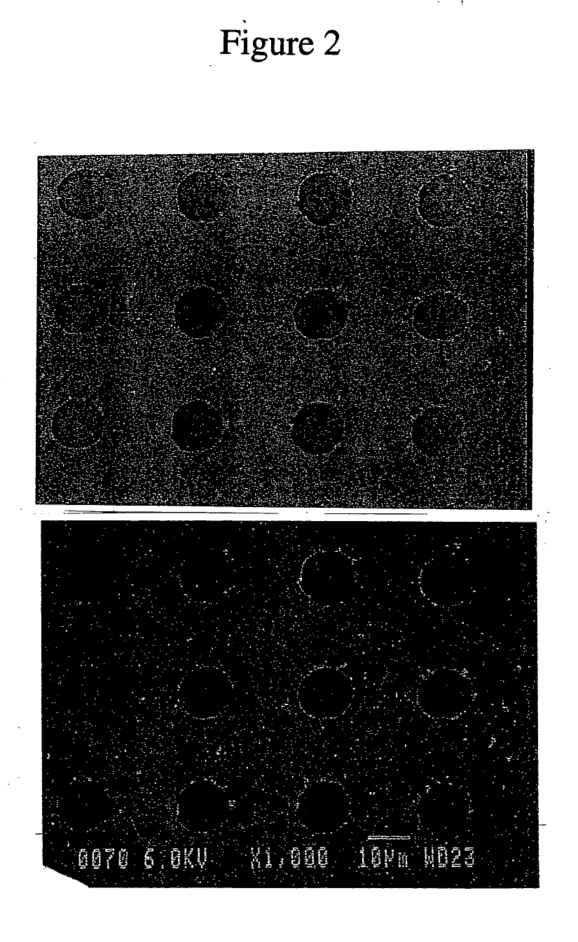

FIGS. 2 & 3 shows scanning electron microscope images of an example of a microcast porous device having 10 micron diameter channels in accordance with the invention.

### Detection Methods

Ordinarily the porous device in accordance with the invention is used in conjunction with a known detection technology particularly adapted to discriminating between bounded regions in which binding has taken place and those in which no binding has occurred and for quantitating the relative extent of binding in different bounded regions. In DNA and RNA sequence detection, autoradiography and optical detection are advantageously used. Alternatively, phosphorimager detection methods may be used.

Optical detection of fluorescent-labeled receptors is also employed in detection. In traditional sequencing, a DNA base-specific fluorescent dye is attached covalently to the oligonucleotide primers or to the chain-terminating dideoxynucleotides used in conjunction with DNA polymerase. The appropriate absorption wavelength for each dye is chosen and used to excite the dye. If the absorption spectra of the dyes are close to each other, a specific wavelength can be chosen to excite the entire set of dyes. One particularly useful optical detection technique involves the use of ethidium bromide, which stains duplex nucleic acids. The fluorescence of these dyes exhibits an approximate twenty-fold increase when it is bound to duplexed DNA or RNA, when compared to the fluorescence exhibited by unbound dye or dye bound to single-stranded DNA. This dye is advantageously used to detect the presence of hybridized polynucleic acids.

A highly preferred method of detection is a charge-coupled-device array or CCD array. With the CCD array, an individual pixel or group of pixels within the CCD array is placed adjacent to each confined region of the substrate where detection is to be undertaken. Light attenuation, caused by the greater absorption of an illuminating light in test sites with hybridized molecules, is used to determine die sites where hybridization has taken place. Lens-based CCD cameras can also be used.

Alternatively, a detection apparatus can be constructed such that sensing of changes in AC conductance or the dissipation of a capacitor placed contiguous to each confined region can be measured. Similarly, by forming a transmission line between two electrodes contiguous to each confined region hybridized molecules can be measured by the radio-frequence (RF) loss. The preferred methods for use herein are described in, Optical and Electrical Methods and Apparatus for Molecule Detection, PCT Published Application WO 93/22678, published Nov. 11, 1993, and expressly incorporated herein by reference.

The disclosed embodiments are by no means exhaustive of the possible formulations or results encompassed by the invention. For this reason, then, reference should be made solely to the appended claims for the purposes of determining the true scope of this invention.

1. A device for binding a target molecule, comprising a polymer substrate having oppositely facing first and second major surfaces; a multiplicity of discrete channels extending through said polymer substrate from said first major surface to said second major surface; a first binding reagent immobilized on the walls of a first group of said channels, and a second binding reagent immobilized on the walls of a second group of said channels.

2. A device according to claim 1, wherein said first and second binding reagents differ from one another.

3. A device according to claim 1, wherein said first and second binding reagents bind different target molecules.

4. A device according to claim 2, comprising discrete channels having diameters of from about 0.033 micrometers to about 10 micrometers.

5. A device according to claim 2, comprising discrete channels having cross sectional areas of between about 8.5×10⁻⁴ m² to 80 μm².

6. A device according to claim 2, comprising a substrate between about 100 μm to about 1000 μm thick.

7. A device according to claim 2, comprising channels having an inner surface area of between about 10 μm² and about 3 μm².

8. A device according to claim 2, wherein said groups of channels have areas of between about 20 μm² to about 3 μm².

9. A device according to claim 2, wherein there are between 400 and 4400 of said groups of discrete channels per cm² of cross-sectional area of the substrate.

10. A device according to claim 2, wherein the inner surface area of the channels in a group of channels is from about 100 to about 1000 times the cross sectional area of the group of channels.

11. A device according to claim 1 wherein said substrate is fabricated from a castable polymer.

12. A device according to claim 11, wherein said castable polymer is an epoxy.

13. A device according to claim 12, wherein said castable polymer is cured by exposure to ultraviolet or near ultraviolet radiation.

14. A device according to claim 1 wherein said binding reagents are effective for carrying out binding reactions selected from the group consisting of binding reactions involving small molecules, macromolecules, particles and cellular systems.

15. A device according to claim 1, wherein said binding reagents are effective for carrying out an analytical task selected from the group consisting of sequence analysis by hybridization, analysis of patterns of gene expression by hybridization of mRNA or cDNA to gene-specific probes, immunochemical analysis of protein mixtures, epitope mapping, assay of receptor-ligand interactions and profiling of cellular populations involving binding of cell surface molecules to specific ligands or receptors.

16. A device according to claim 15, wherein said binding reagents are selected from the group consisting of DNA, proteins and ligands.

17. A device according to claim 16, wherein said binding reagents are oligonucleotide probes.

18. A device according to claim 1, further comprising a support, wherein said support is integral to said substrate, or is bonded to said substrate.

19. A device according to claim 18 wherein said support is integral to said substrate.

20. A device according to claim 18, wherein said support is bonded to said substrate.

21. A device according to claim 18, wherein said support comprises wells or channels for delivering fluids to subsets of channels of said substrate.

22. A device according to claim 20 wherein said support is made from a castable polymer.

23. A device according to claim 1 wherein said polymer substrate has a lower specific stiffness than glass or silicon

24. A device according to claim 20 wherein said polymer substrate has a lower specific stiffness than glass or silicon

25. A device for binding a target molecule, comprising a substrate having oppositely facing first and second major surfaces; a multiplicity of discrete channels extending through said substrate from said first major surface to said second major surface; a first binding reagent immobilized on the walls of a first group of said channels, and a second binding reagent immobilized on the walls of a second group of said channels; the improvement comprising a substrate made from a polymer.
